(19) United States
(12) Patent Application Publication
(10) Pub. No.: US 2025/0186797 A1
(43) Pub. Date: Jun. 12, 2025

(54) EYEWEAR-BASED DEVICE FOR IMPROVED BLOOD CIRCULATION AND THERAPY AROUND EYES

(71) Applicant: FARID LAKHANI, San Antonio, TX (US)

(72) Inventor: FARID LAKHANI, San Antonio, TX (US)

(21) Appl. No.: 18/970,658

(22) Filed: Dec. 5, 2024

Related U.S. Application Data
(60) Provisional application No. 63/606,843, filed on Dec. 6, 2023.

(51) Int. Cl. A61N 2/00; A61N 2/06

(52) U.S. Cl. CPC A61N 2/004; A61N 2/06

(57) ABSTRACT

Embodiments provide for an apparatus comprising an eyewear-based or headwear-based wearable device (“device”) having installed a band coupled to a magnet to generate magnetic field to facilitate regulation of blood flow and therapeutic benefits to a user wearing the device.

### CROSS-REFERENCE

This application claims priority to U.S. Provisional Application No. 63/606,843, filed on Dec. 6, 2023, which is hereby incorporated herein by reference.

### FIELD

One or more implementations relate generally to eyewear and more specifically, to an eyewear-based device for improved blood circulation and therapy around eyes.

### BACKGROUND

Although numerous wearable comforting devices are available, most conventional wearable comforting devices are limited in terms of their basic structure and lack the flexibility, convenience, and results that users would prefer to have when wearing a device on their faces or around their eyes.

### BRIEF DESCRIPTION OF THE DRAWINGS

In the following drawings, like reference numbers are used to refer to like elements. Although the following figures depict various examples, one or more implementations are not limited to the examples depicted in the figures.

FIG. 1 illustrates an architectural structure of an eyewear-based wearable device with a series of magnets capable of being installed on each hand of the eyewear-based wearable device for flexibility, comfort, and convenience in terms of improved blood circulation and facial therapy around eyes according to one embodiment.

FIG. 2 illustrates practical implementation of an eyewear-based wearable device with multiple magnets on each side according to one embodiment.

FIG. 3 illustrates a bottom view of an eyewear-based wearable device with multiple magnets on each side for flexibility, comfort, and convenience in terms of improved blood circulation and facial therapy around eyes according to one embodiment.

FIG. 4 illustrates a top view of an eyewear-based wearable device with multiple magnets on each side for flexibility, comfort, and convenience in terms of improved blood circulation and facial therapy around eyes according to one embodiment.

FIG. 5 illustrates a view of a series of multiple magnets on a rubber- or plastic-based material to be installed on each side of an eyewear-based wearable device for flexibility, comfort, and convenience in terms of improved blood circulation and facial therapy around eyes according to one embodiment.

FIG. 6 illustrates another view of a series of multiple magnets on a rubber- or plastic-based material to be installed on each side of an eyewear-based wearable device for flexibility, comfort, and convenience in terms of improved blood circulation and facial therapy around eyes according to one embodiment.

FIG. 7 illustrates a view of a series of multiple magnets on a rubber- or plastic-based material installed on one hand of an eyewear-based wearable device for flexibility, comfort, and convenience in terms of improved blood circulation and facial therapy around eyes according to one embodiment.

FIG. 8 illustrates another view of a series of multiple magnets on a rubber- or plastic-based material installed on both hands of an eyewear-based wearable device for flexibility, comfort, and convenience in terms of improved blood circulation and facial therapy around eyes according to one embodiment.

FIG. 9 illustrates a view of a series of multiple magnets on a rubber- or plastic-based material installed on one hand of an eyewear-based wearable device for flexibility, comfort, and convenience in terms of improved blood circulation and facial therapy around eyes according to one embodiment.

FIG. 10 illustrates a view of a series of multiple magnets on a rubber- or plastic-based material installed on one hand of an eyewear-based wearable device for flexibility, comfort, and convenience in terms of improved blood circulation and facial therapy around eyes according to one embodiment.

FIG. 11 illustrates a view of a series of multiple magnets on a rubber- or plastic-based material installed on both hands an eyewear-based wearable device, where both hands are neatly folded when the eyewear-based wearable device is not in use, according to one embodiment.

FIG. 12 illustrates a view of a series of multiple magnets on a rubber- or plastic-based material installed on one hand of an eyewear-based wearable device for flexibility, comfort, and convenience in terms of improved blood circulation and facial therapy around eyes according to one embodiment.

FIG. 13 illustrates practical implementation of an eyewear-based wearable device with multiple magnets installed on each hand of the eyewear-based wearable device such that the multiple magnets are toughing the temple area and skin of a user wearing the eyewear-based wearable device according to one embodiment.

FIG. 14 illustrates a practical implementation of an eyewear-based wearable device with multiple magnets installed on each hand of the eyewear-based wearable device such that the multiple magnets are toughing the temple area and skin of a user wearing the eyewear-based wearable device according to one embodiment.

FIG. 15 illustrates a view of a bar of magnet on a rubber- or plastic-based material installed on one hand of an eyewear-based wearable device for flexibility, comfort, and convenience in terms of improved blood circulation and facial therapy around eyes according to one embodiment.

FIG. 16 illustrates a view of a bar of magnet on a rubber- or plastic-based material installed on one hand of an eyewear-based wearable device for flexibility, comfort, and convenience in terms of improved blood circulation and facial therapy around eyes according to one embodiment.

FIG. 17 illustrates a view of a bar of magnet on a rubber- or plastic-based material installed on one hand of an eyewear-based wearable device for flexibility, comfort, and convenience in terms of improved blood circulation and facial therapy around eyes according to one embodiment.

FIG. 18 illustrates an architectural structure of an eyewear-based wearable device with a bar of magnet capable of being installed on each hand of the eyewear-based wearable device for flexibility, comfort, and convenience in terms of improved blood circulation and facial therapy around eyes according to one embodiment.

FIG. 19 illustrates a view of a bar of magnet on a rubber- or plastic-based material capable of being installed on each hand of an eyewear-based wearable device for flexibility, comfort, and convenience in terms of improved blood circulation and facial therapy around eyes according to one embodiment

FIG. 20 illustrates a view of a bar of magnet on a rubber- or plastic-based material capable of being installed on each hand of an eyewear-based wearable device for flexibility, comfort, and convenience in terms of improved blood circulation and facial therapy around eyes according to one embodiment

FIG. 21 illustrates a practical implementation of an eyewear-based wearable device with a bar of magnet installed on each hand of the eyewear-based wearable device such that the bars of magnet are toughing the temple area and skin of a user wearing the eyewear-based wearable device according to one embodiment.

### DETAILED DESCRIPTION

In the following description, numerous specific details are set forth. However, embodiments of the invention may be practiced without these specific details. In other instances, well-known circuits, structures, and techniques have not been shown in detail in order not to obscure the understanding of this description.

Embodiments offer an eyewear-based wearable device (“novel eyewear” or “novel therapy device” or simply “eyewear”), also referenced as “Magnetismo” or “Mag-Eye”, that is a cutting-edge eyewear-based device that can be worn by a user having coupled to their eyewear (e.g., eyeglasses such as vision eyeglasses, sunglasses, protective eyeshades, etc.), where this novel therapy device allows for creating a mild magnetic field to induce small blood vessels of the user to dilate or constrict to facilitate regulation of blood flow (such as increased or decreased or constant blood flow) in the impacted area (such as the head area around the eyes) to offer benefits, such as reduced stiffness, inflammation, and overall pain (like headaches).

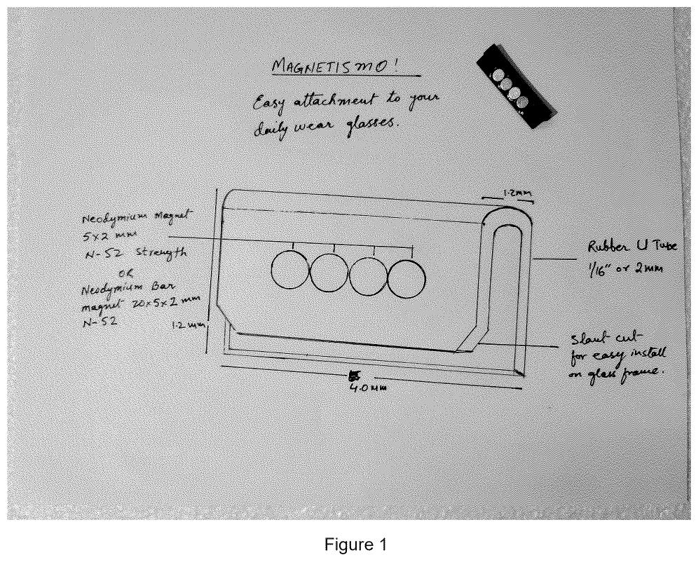

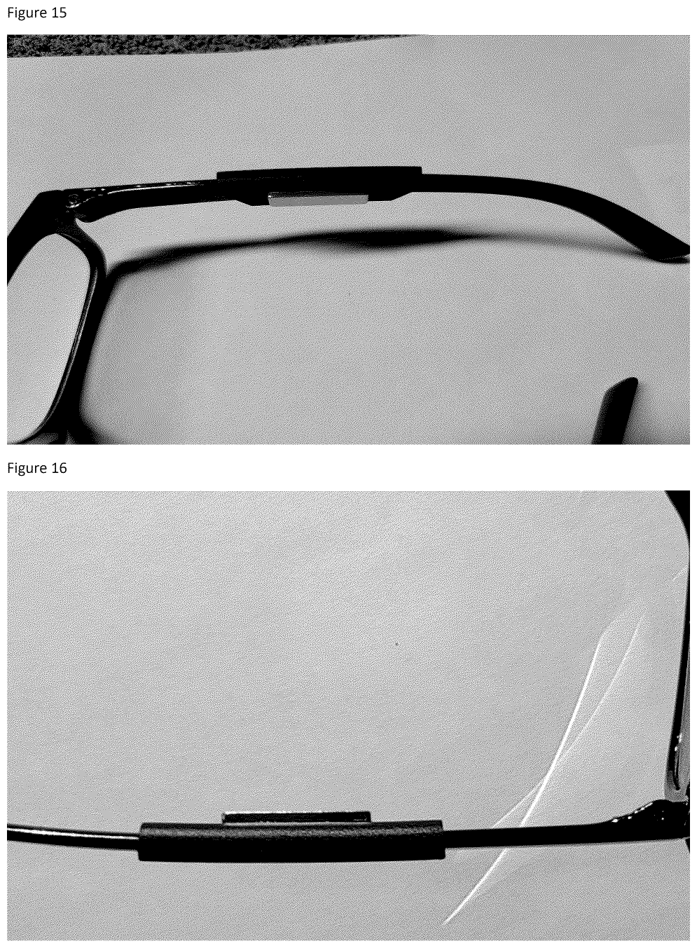

In one embodiment, this eyewear consists of an attachment, such as a U-shaped, attachment, that is made out of an agreeable material, such as rubber, that does not irritate the skin, while one or more magnets, such as one or more neodymium therapy magnets, are placed on the sides that are allowed to touch the user's skin around the head/eye area for maximum conform and benefits. In one embodiment, a series of magnets may be employed on each side as illustrated in FIGS. 1-14, while, in another embodiment, a single bar of magnet may be employed on each side as illustrated in FIGS. 15-21. It is to be noted and appreciated that embodiments are not limited to any number or types or forms or powers of magnets used for or employed with the novel therapy device. Further, in one embodiment, this novel therapy device is convenient in that it is capable of being coupled to both stems of any type or form or function of eyewear (such as sliding on both stems of eyeglasses) worn for vision correction, shade, therapeutic reasons, or simply fashion. Further, this novel therapy device's molded shape and slant cuts on the edges make it easier to slide on to the stems of any eyewear and keep the associated magnets securely placed on temples of the head region around the eyes of a user.

In one embodiment, this novel therapy device optimizes widely acceptable form of therapy using static magnets, where this novel therapy device in its application and use offer a great deal and level of therapeutic assistance to those individuals who spend extended amount of time in front of display screens or get frequent headaches due to stressed nerves and poor blood circulation around the eyes. This novel therapy device facilitates proper levels of blood flow and oxygen in the region around the eyes of a user to prevent tissue damage that can result in poor eyesight or even vision loss.

FIG. 1 illustrates an architectural structure of an eyewear-based wearable device with a series of magnets capable of being attached to a band and installed on each hand of the eyewear-based wearable device for flexibility, comfort, and convenience in terms of improved blood circulation and facial therapy around eyes according to one embodiment.

As illustrated, in one embodiment, multiple (such as 2, 3, or 4, or more) relatively small magnets are attached to band that is made of leather, rubber, plastic, or other similar material that is then capable of being installed on one or both hands of an eyewear. It contemplated, and as an example illustrated in FIG. 1, these magnets are not limited to a particular type or form of magnets. For example, these magnets could be neodymium magnets of N-52 strength or of any other suitable type of magnet, and similarly, these magnets could be round, oval, square, rectangular, or any other suitable shape and size. Similarly, as illustrated in FIG. 1, the band could be of any suitable material, such as rubber, and shaped in a manner, such as U-shaped, for easy, durable, and effective installation on the hands of the eyewear.

As aforementioned, this use of magnets on eyewear or other headwear devices, allows for creating a mild magnetic field to induce small blood vessels of the user (wearing the eyewear or other similar device) to dilate or constrict to facilitate regulation of blood flow (such as increased or decreased or constant blood flow) in the impacted area (such as temple or and other the head area around the eyes) to offer benefits, such as significant reduction in stiffness, inflammation, and overall pain (like headaches). In one embodiment, this is accomplished through magnetic field generated by the magnets conveniently touching the user's skin via the eyewear and without having to invest in any other expensive therapies or elaborate contraptions.

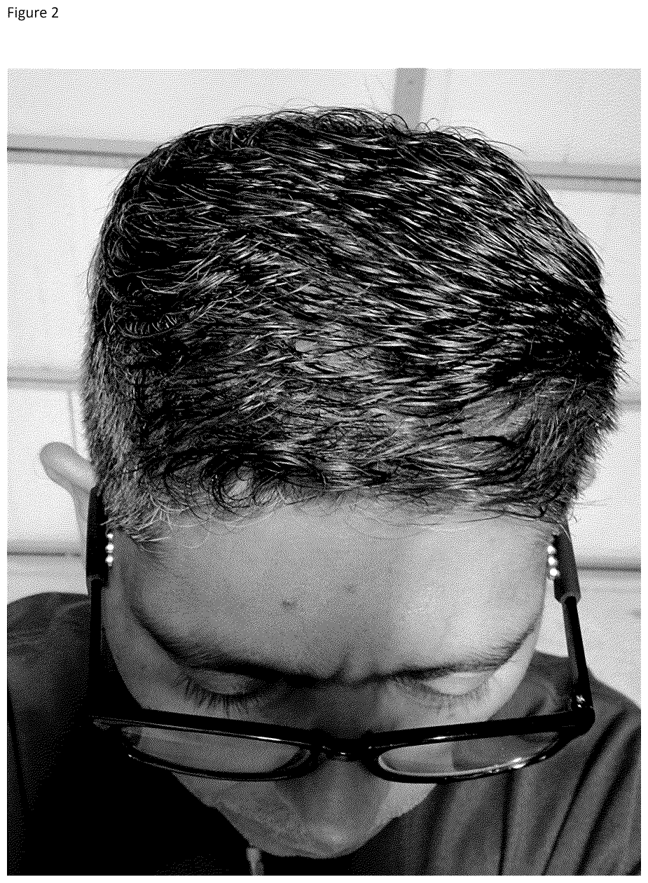

FIG. 2 illustrates practical implementation of an eyewear-based wearable device with multiple magnets on each side according to one embodiment. In one embodiment, FIG. 2 illustrates how the magnets conveniently and comfortably touch the user's temporal or head area around the eyes providing instant therapy and relief from pain to the user.

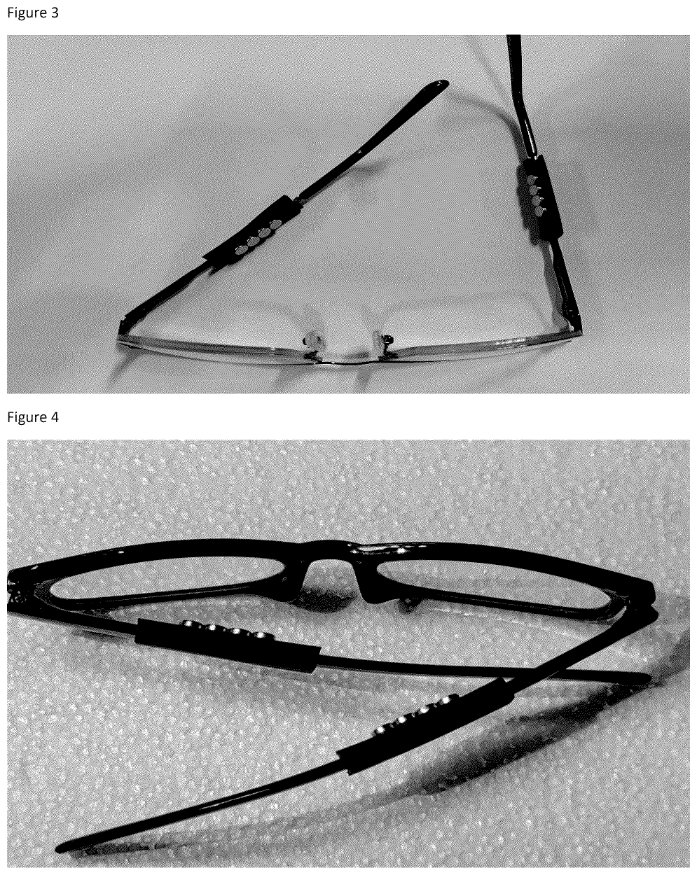

FIG. 3 illustrates a bottom view of an eyewear-based wearable device with multiple magnets on each side for flexibility, comfort, and convenience in terms of improved blood circulation and facial therapy around eyes according to one embodiment. In this embodiment, the series of magnets are installed via two bands, each band around a hand of the eyewear such that the magnets are balanced and point to the user's temporal area.

FIG. 4 illustrates a top view of an eyewear-based wearable device with multiple magnets on each side for flexibility, comfort, and convenience in terms of improved blood circulation and facial therapy around eyes according to one embodiment. In this embodiment, the hands of the eyewear are folded when not in use, but the bands remain installed on the hands as the bands are designed such that they do not get in the way of folding the hands when the eyewear is not in use. In another embodiment, the illustrated bands may be removed when the eyewear is not in use.

FIG. 5 illustrates a view of a series of multiple magnets on a rubber- or plastic-based material to be installed on each side of an eyewear-based wearable device for flexibility, comfort, and convenience in terms of improved blood circulation and facial therapy around eyes according to one embodiment. As previously discussed, there are restrictions or limitations as to the size or shape or number of magnets or the type or shape of material used for the band. In one embodiment, the magnets and the band are kept small and lightweight and are made from materials that are best suited for the user and most effective in offering comfort and therapy to the user.

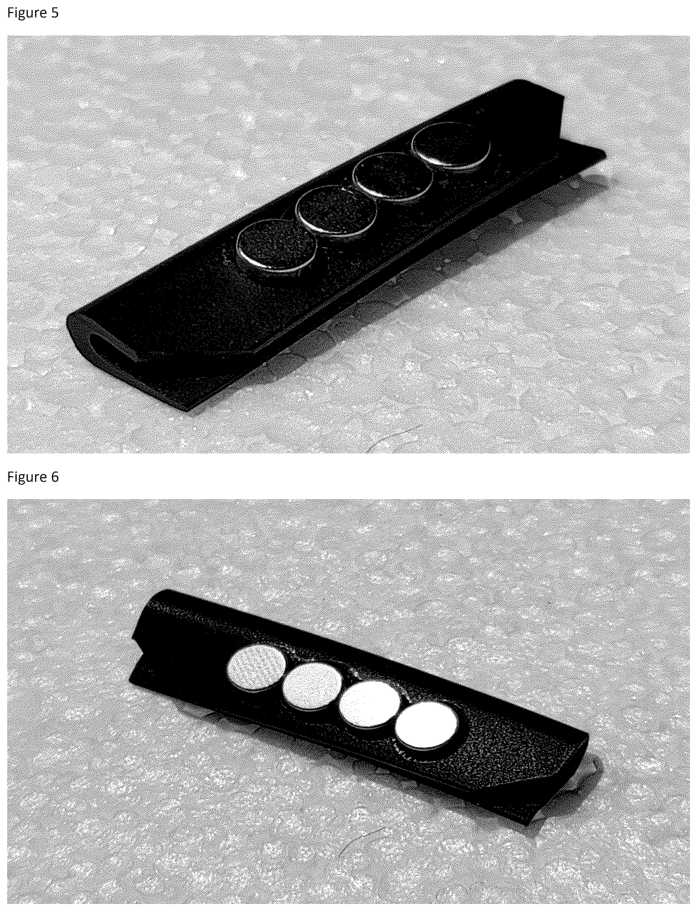

FIG. 6 illustrates another view of a series of multiple magnets on a rubber-based band capable of being installed on a hand of an eyewear-based wearable device for flexibility, comfort, and convenience in terms of improved blood circulation and facial therapy around eyes according to one embodiment.

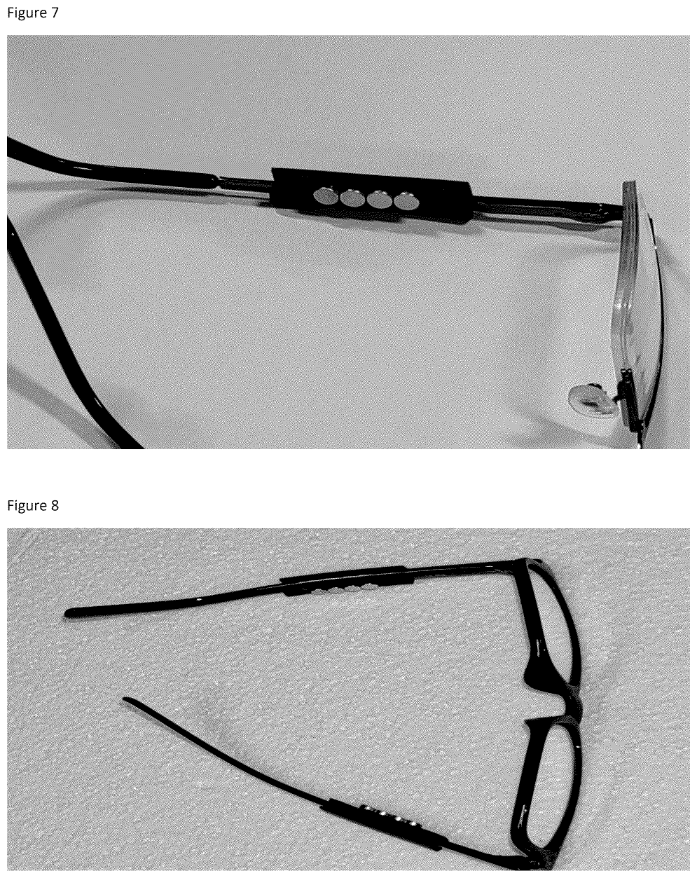

FIG. 7 illustrates a view of a series of multiple magnets attached to a rubber-based band installed on one hand of an eyewear-based wearable device for flexibility, comfort, and convenience in terms of improved blood circulation and facial therapy around eyes according to one embodiment.

FIG. 8 illustrates another view of a series of multiple magnets attached to two rubber-based bands shown as installed on both hands of an eyewear-based wearable device for flexibility, comfort, and convenience in terms of improved blood circulation and facial therapy around eyes according to one embodiment.

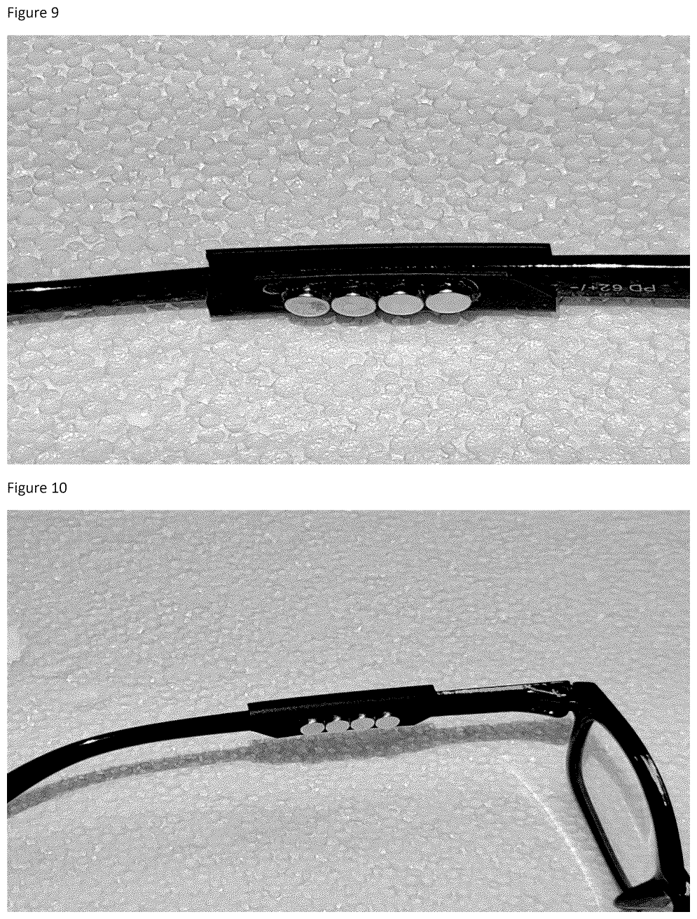

FIG. 9 illustrates a view of a series of multiple magnets attached to a rubber-based band installed on one hand of an eyewear-based wearable device for flexibility, comfort, and convenience in terms of improved blood circulation and facial therapy around eyes according to one embodiment.

FIG. 10 illustrates a view of a series of multiple magnets attached on a rubber-based installed on one hand of an eyewear-based wearable device for flexibility, comfort, and convenience in terms of improved blood circulation and facial therapy around eyes according to one embodiment.

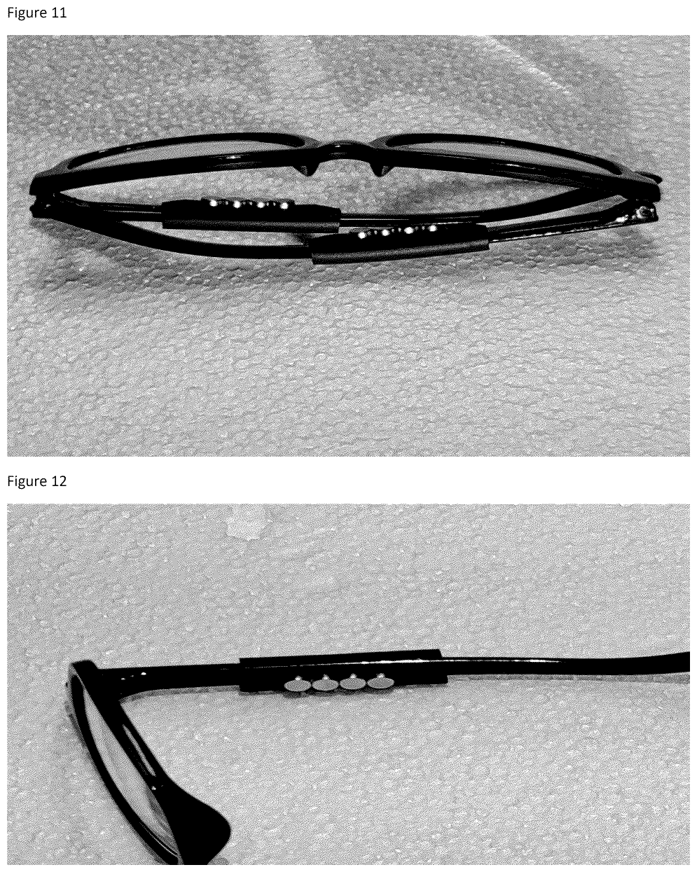

FIG. 11 illustrates a view of a series of multiple magnets attached to a rubber-based band installed on both hands an eyewear-based wearable device, where both hands are neatly folded when the eyewear-based wearable device is not in use and where the two bands remain installed on the two respective hands, according to one embodiment.

FIG. 12 illustrates a view of a series of multiple magnets attached to a rubber-based band installed on one hand of an eyewear-based wearable device for flexibility, comfort, and convenience in terms of improved blood circulation and facial therapy around eyes according to one embodiment.

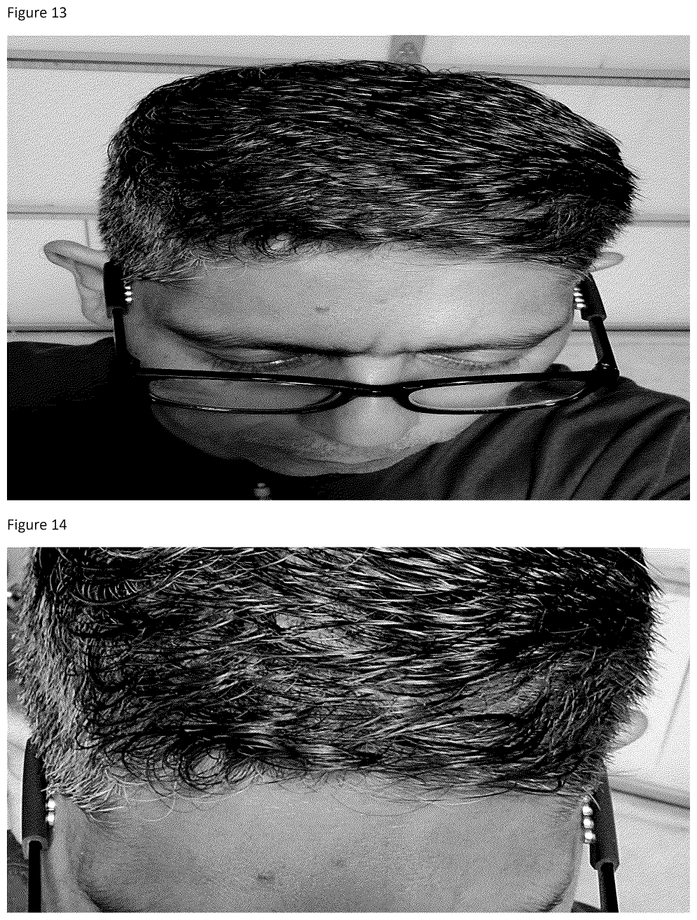

FIG. 13 illustrates a practical implementation of an eyewear-based wearable device with multiple magnet bands installed on both hand of the eyewear-based wearable device such that the multiple magnets are conveniently and comfortable touching the temple area and head skin of a user wearing the eyewear-based wearable device according to one embodiment.

FIG. 14 illustrates yet another practical implementation of an eyewear-based wearable device with multiple magnet bands installed on both hand of the eyewear-based wearable device such that the multiple magnets are conveniently and comfortable touching the temple area and head skin of a user wearing the eyewear-based wearable device according to one embodiment.

FIG. 15 illustrates a view of a bar of magnet attached to a rubber-based band installed on one hand of an eyewear-based wearable device for flexibility, comfort, and convenience in terms of improved blood circulation and facial therapy around eyes according to one embodiment.

As illustrated and described previously with respect to FIG. 1, magnets and band are not limited in terms of their shape, size, or material. Accordingly, here, in FIG. 15, is illustrated another embodiment where instead of multiple magnets of FIG. 1, a single rectangular bar like magnets is attached to a rubber-based band that is then similarly installed on a hand of an eyewear. Having smaller multiple magnets or a singular bar of magnet may simply be a matter of choice for the users as in terms of their effectiveness and benefits, the two embodiments may provide virtually equal levels of therapy, comfort, and convenience to the users.

As described earlier, this bar of magnet may be a neodymium magnet of N-52 strength or of any other suitable type of magnet, and similarly, the magnet may be rectangular, square, oval, round, or any other suitable shape. Given that is bar of magnet is a single bar providing the same benefit, in one embodiment, it is likely to be bigger in size than each of the series of multiple magnets of FIG. 1; nevertheless, the bar of magnet of FIG. 15 is not limited to a specific size. Similarly, as illustrated in FIG. 1, the band that holds the bar of magnet could be of any suitable material, such as rubber, and shaped in a manner, such as U-shaped, for easy, durable, and effective installation on the hands of the eyewear.

FIG. 16 illustrates a view of a bar of magnet attached to a rubber-based bar installed on one hand of an eyewear-based wearable device for flexibility, comfort, and convenience in terms of improved blood circulation and facial therapy around eyes according to one embodiment.

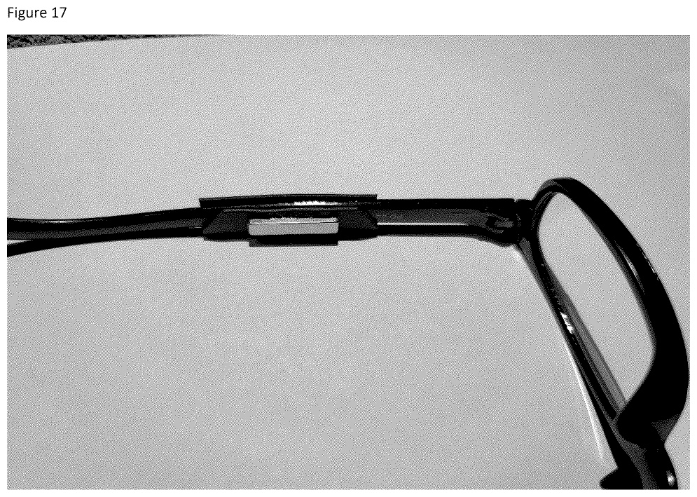

FIG. 17 illustrates a view of a bar of magnet attached to a rubber-based bar installed on one hand of an eyewear-based wearable device for flexibility, comfort, and convenience in terms of improved blood circulation and facial therapy around eyes according to one embodiment.

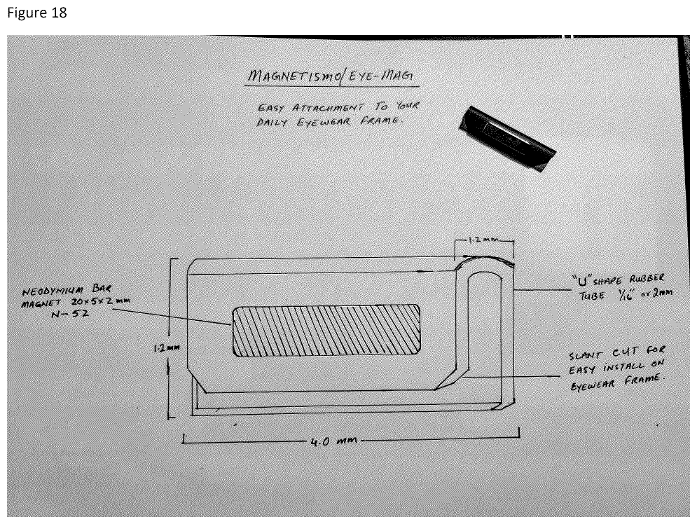

FIG. 18 illustrates an architectural structure of an eyewear-based wearable device with a bar of magnet capable of being attached to a band and installed on each hand of the eyewear-based wearable device for flexibility, comfort, and convenience in terms of improved blood circulation and facial therapy around eyes according to one embodiment.

As described earlier, this bar of magnet may be a neodymium magnet of N-52 strength or of any other suitable type of magnet, and similarly, the magnet may be rectangular, square, oval, round, or any other suitable shape. Given that is bar of magnet is a single bar providing the same benefit, in one embodiment, it is likely to be bigger in size than each of the series of multiple magnets of FIG. 1; nevertheless, the bar of magnet of FIG. 18 is not limited to a specific size. Similarly, as illustrated in FIG. 1, the band that holds the bar of magnet could be of any suitable material, such as rubber, and shaped in a manner, such as U-shaped, for easy, durable, and effective installation on the hands of the eyewear

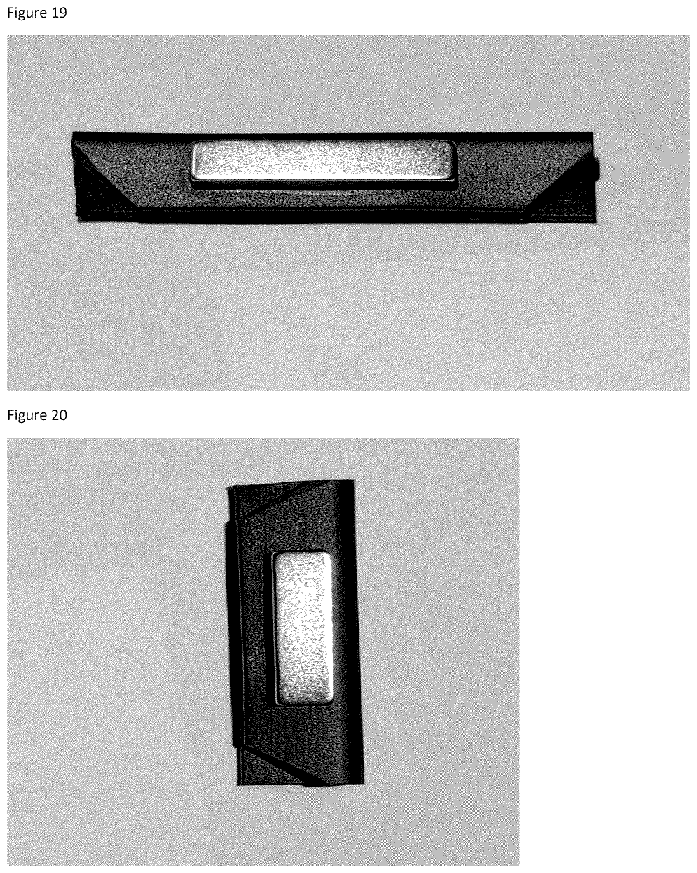

FIG. 19 illustrates a view of a bar of magnet attached to a rubber-based band capable of being installed on each hand of an eyewear-based wearable device for flexibility, comfort, and convenience in terms of improved blood circulation and facial therapy around eyes according to one embodiment

FIG. 20 illustrates a view of a bar of magnet attached to a rubber-based band capable of being installed on each hand of an eyewear-based wearable device for flexibility, comfort, and convenience in terms of improved blood circulation and facial therapy around eyes according to one embodiment

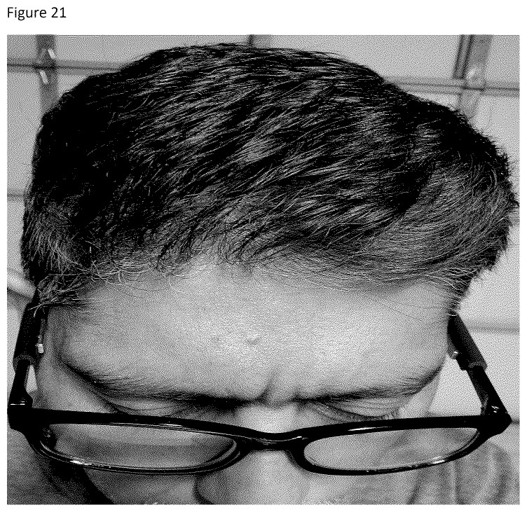

FIG. 21 illustrates a practical implementation of an eyewear-based wearable device with a bar of magnet attached to a band installed on each hand of the eyewear-based wearable device such that the bars of magnet are toughing the temple area and skin of a user wearing the eyewear-based wearable device according to one embodiment.

Any of the above embodiments may be used alone or together with one another in any combination. Embodiments encompassed within this Specification may also include embodiments that are only partially mentioned or alluded to or are not mentioned or alluded to at all in this brief summary or in the abstract. Although various embodiments may have been motivated by various deficiencies with the prior art, which may be discussed or alluded to in one or more places in the specification, the embodiments do not necessarily address any of these deficiencies. In other words, different embodiments may address different deficiencies that may be discussed in the specification. Some embodiments may only partially address some deficiencies or just one deficiency that may be discussed in the specification, and some embodiments may not address any of these deficiencies.

While one or more implementations have been described by way of example and in terms of the specific embodiments, it is to be understood that one or more implementations are not limited to the disclosed embodiments. To the contrary, it is intended to cover various modifications and similar arrangements as would be apparent to those skilled in the art. Therefore, the scope of the appended claims should be accorded the broadest interpretation so as to encompass all such modifications and similar arrangements. It is to be understood that the above description is intended to be illustrative, and not restrictive.

What is claimed is:

1. An apparatus comprising:
an eyewear-based or headwear-based wearable device (“device”) having installed a band coupled to a magnet to generate magnetic field to facilitate regulation of blood flow and therapeutic benefits to a user wearing the device.

2. The apparatus of claim 1, wherein the magnet comprises multiple magnets coupled to the band, wherein the multiple magnets include multiple neodymium therapy magnets.

3. The apparatus of claim 1, wherein the magnet comprises a single magnet including a bar of magnet, wherein the bar of magnet include a bar of multiple neodymium therapy magnet.

4. The apparatus of claim 1, wherein the band comprises a U-shaped band capable of being installed on a hand or an extension or a stem of the device such that the magnet touches the skin of the user, wherein the skin includes a temporal or an area near an eye of the user, wherein the band is made of one or more of rubber, plastic, leather, or metal.

5. A system comprising:
an eyewear-based or headwear-based wearable system (“system”) having installed a band coupled to a magnet to generate magnetic field to facilitate regulation of blood flow and therapeutic benefits to a user wearing the system.

6. The system of claim 5, wherein the magnet comprises multiple magnets coupled to the band, wherein the multiple magnets include multiple neodymium therapy magnets.

7. The system of claim 5, wherein the magnet comprises a single magnet including a bar of magnet, wherein the bar of magnet include a bar of multiple neodymium therapy magnet.

8. The system of claim 5, wherein the band comprises a U-shaped band capable of being installed on a hand or an extension or a stem of the system such that the magnet touches the skin of the user, wherein the skin includes a temporal or an area near an eye of the user, wherein the band is made of one or more of rubber, plastic, leather, or metal.

9. A method comprising:
facilitating an eyewear-based or headwear-based wearable device (“device”) having installed a band coupled to a magnet to generate magnetic field to facilitate regulation of blood flow and therapeutic benefits to a user wearing the device.

10. The method of claim 9, wherein the magnet comprises multiple magnets coupled to the band, wherein the multiple magnets include multiple neodymium therapy magnets.

11. The method of claim 9, wherein the magnet comprises a single magnet including a bar of magnet, wherein the bar of magnet include a bar of multiple neodymium therapy magnet.

12. The method of claim 9, wherein the band comprises a U-shaped band capable of being installed on a hand or an extension or a stem of the device such that the magnet touches the skin of the user, wherein the skin includes a temporal or an area near an eye of the user, wherein the band is made of one or more of rubber, plastic, leather, or metal.
